(19) United States
(12) Patent Application Publication
(10) Pub. No.: US 2009/0197938 A1
(43) Pub. Date: Aug. 6, 2009

(54) Polyphenol Type Compounds, Compositions Containing Same and Use Thereof for Preventing or Treating Diseases Involving Abnormal Cell Proliferation

(75) Inventors: Gaëlle Depierre, Saint-Seurin-de-Bourg (FR); Stephane Quideau, Talence (FR); Michael Jourdes, Pranzac (FR); Patrick Pardon, Villenave d'Ornon (FR); Philippe Pourquier, Beychac-et-Caillau (FR)

Correspondence Address: HARNESS, DICKEY & PIERCE, P.L.C., P.O. BOX 828, BLOOMFIELD HILLS, MI, 48303, US

(73) Assignees: Fluofarma, Pessac (FR); UNIVERSITE DE BORDEAUX 1, Talence Cedex (FR)

(21) Appl. No.: 11/922,516

(22) PCT Filed: Jun. 23, 2006
(86) PCT No.: PCT/FR2006/001455

§ 371 Date: Mar. 13, 2009

(30) Foreign Application Priority Data
Jul. 1, 2005 (FR) 0507051

(51) Int. Cl. A61K 31/365; C07D 323/00; A61P 35/00

(52) U.S. Cl. 514/450; 549/267

(57) ABSTRACT

The invention concerns compounds of formula (I), wherein —XₘR₁ and YₙR₂, identical or different are present, or one of the XₘR₁ or YₙR₂ is absent and YₐR₂, identical or different, is present, and in that case the bond between C₁ and C₂ is a double bond, XₘR₁ and YₙR₂ together represent a group A and in that case the bond between C₁ and A is a double bond, or their pharmaceutically acceptable salts as medicine.

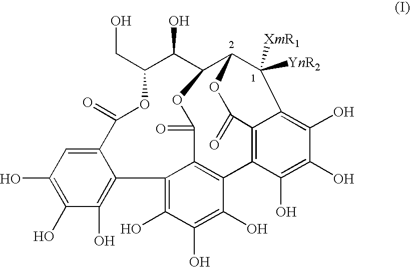

The present invention concerns polyphenol type compounds, in particular for drugs, and the use of such compounds in the preparation of pharmaceutical compositions. The invention also concerns compositions comprising compounds of this type, or even the use of compounds of this type in the preparation of pro-apoptotic compositions. The compositions may, in particular, be used for the preparation or treatment of diseases involving abnormal cell proliferation, for example cancer.

Cancer is one of the largest causes of mortality and, as a result, currently one of the most serious public health issues in the world. In this respect, a great many drugs have been developed, unfortunately, they are not able to successfully treat all cases. In particular, resistances to certain antineoplastic agents have been discovered. It is therefore important to find new therapeutic means and set up new treatment strategies.

Several antitumor agents are inhibitors of DNA topoisomerase II. However, certain compounds that inhibit DNA topoisomerase II are also known to stimulate the proliferation of cancer cells with subtoxic concentrations. This has been described for certain compounds from the family of anthracyclines by P. Vichi and T. R. Tritton, Cancer Res., 1989, 49(10), 2679-82, and by M. G. Thompson and J. A. Hickman, Eur. J. Cancer, 1991, (27)10, 1263-8.

Certain polyphenol compounds of the ellagitannin C-glycoside type are DNA topoisomerase II inhibitors. Among them, certain compounds from the family of ellagitannin C-glycosides comprise a polyphenol nonahydroxyterphenol (NHTP) radical and their derivatives, for example, vescalagine, castalagine, acutissimine A and B, and epiacutissamine A and B, stimulate the proliferation of cancer cells with subtoxic doses.

It is clear that this effect is especially bothersome in the use of these compounds in the fight against diseases involving abnormal cell proliferation.

Therefore, one of the goals of the invention is to provide new compounds that inhibit DNA topoisomerase II presenting an improved antineoplastic action, in particular related to a pro-apoptotic activity and in particular not favouring the proliferation of cancer cells with subtoxic doses.

The inventors have now discovered that compounds such as those defined below are DNA topoisomerase II inhibitors and present an antineoplastic activity, in particular related to a pro-apoptotic activity and do not increase or barely increase the proliferation of cancer cells, in particular with subtoxic doses.

Therefore, the object of the invention comprises compounds complying with formula (I), or its pharmaceutically acceptable salts, as a drug:

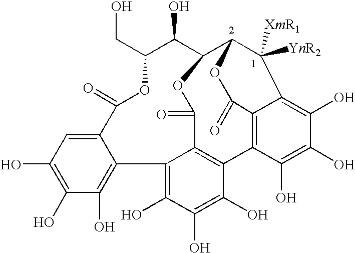

### Formula (I)

wherein
XₘR₁, and YₙR₂, identical or different, are present, or
one of XₘR₁, and YₙR₂ is absent, and in this case the bond between C₁ and C₂ is a double bond, or
XₘR₁ and YₙR₂ together represent a group A and in this case the bond between C₁ and A is a double bond, where:
R₁ and R₂, independent of one another, represent an atom of hydrogen; an atom of halogen; an alkyl, alkene or alkyne radical, comprising 1 to 18 atoms of carbon, possibly substituted by one or several amino, acid, acid derivative, alkoxy, aryl or hydroxy groups; an aryl radical possibly substituted by one or several amino, acid, acid derivative, alkoxy, aryl or hydroxy groups; an arylalkyl or alkylaryl radical, possibly substituted by one or several amino, acid, acid derivative, alkoxy, aryl or hydroxy groups; or a nitro function;
   or R₁, and R₂ together form a cycle or a heterocycle, possibly substituted,
m is a whole number chosen among 0 and 1, n is a whole number chosen among 0 and 1,
X and Y represent independent of each other —O—, —OC(═O)—, —OC(═O)O—, —OC(═O)N—, —NR₃—, —NR₃C(═O)—, —NR₃C(═O)O—, —NR₃C(O)NR₃—, —C(═O)—, —C(═O)O—, —S(O)_{q}—, or —P(O)ᵣ—,
q is a whole number chosen among 0, 1, 2 and 3, r is a whole number chosen among 0, 1, 2 and 3,
R₃ represents an atom of hydrogen; an alkyl, alkene or alkyne radical, comprising from 1 to 18 carbons, possibly substituted by one or several amino, acid, acid derivative, alkoxy, aryl or hydroxy groups; an aryl radical, possibly substituted by one or several amino, acid, acid derivative, alkoxy, aryl or hydroxy groups; an arylalkyl or alkylaryl radical possibly substituted by one or several amino, acid, acid derivative, alkoxy, aryl or hydroxy groups; or an alkoxy radical possibly substituted by an aryl radical;
A represents O, S, NOR₅, NR₆ or CR₅R₆, where R₅ and R₆ represent independent of each other an atom of hydrogen, an alkyl, alkene or alkyne radical, comprising from 1 to 18 atoms of carbon, possibly substituted by one or several amino, acid, acid derivative, alkoxy, aryl or hydroxy groups; an arylalkyl or alkylaryl radical possibly substituted by one or several amino, acid, acid derivative, alkoxy, aryl or hydroxy groups; or an alkoxy radical, possibly substituted by an aryl radical.

Of course, the compounds according to the invention may also be optical and geometric isomers, in particular at the carbon I level, compounds defined above, as well as their racemic mixtures.

Preferred compounds according to the invention comply with formula (I), or one of its pharmaceutically acceptable salts:

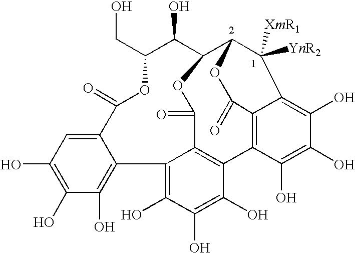

### Formula (I)

wherein:
R₁ and R₂ represent, independent of one another, an atom of hydrogen; an atom of halogen; an alkyl, alkene or alkyne radical, containing 1 to 18 atoms of carbon, possibly substituted by one or several amino, acid, acid derivative, alkoxy, aryl or hydroxy groups; an aryl radical possibly substituted by one or several amino, acid, acid derivative, alkoxy, aryl or hydroxy groups; an arylalkyl or alkylaryl radical, possibly substituted by one or several amino, acid, acid derivative, alkoxy, aryl or hydroxy groups; or a nitro function;
   or R₁ and R₂ together form a cycle or a heterocycle, possibly substituted,
m is a whole number chosen among 0 and 1, n is a whole number chosen among 0 and 1,
X and Y represent independent of each other —O—, —OC(═O)—, —OC(═O)O—, —OC(═O)N—, —NR₃—, —NR₃C(═O)—, —NR₃C(═O)O—, —NR₃C(O)NR₃—, —C(═O)—, —C(═O)O—, —S(O)_{q}—, or —P(O)ᵣ—,
q is a whole number chosen among 0, 1, 2 and 3, r is a whole number chosen among 0, 1, 2 and 3,
R₃ represents an atom of hydrogen; an alkyl, alkene or alkyne radical, comprising 1 to 18 atoms of carbon, possibly substituted by one or several amino, acid, acid derivative, alkoxy, aryl or hydroxy groups; an aryl radical, possibly substituted by one or several amino, acid, acid derivative, alkoxy, aryl or hydroxy groups; an arylalkyl or alkylaryl radical possibly substituted by one or several amino, acid, acid derivative, alkoxy, aryl or hydroxy groups; or an alkoxy radical possibly substituted by an aryl radical.

Other preferred compounds according to the invention comply with formula (II) or one of its pharmaceutically acceptable salts:

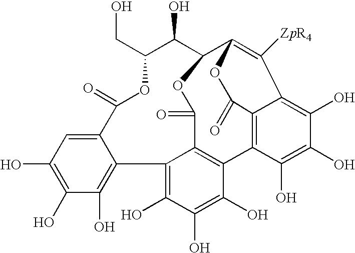

### Formula (II)

wherein:
p is a whole number chosen among 0 and 1,
z represents —O—, —OC(═O)—, —OC(═O)O—, —OC(═O)N—, —NR₃—, —NR₃C(═O)—, —NR₃C(═O)O—, —NR₃C(O)NR₃—, —C(═O)—, —C(═O)O—, —S(O)_{q}—, or —P(O)ᵣ—,
q is a whole number chosen among 0, 1, 2 and 3, r is a whole number chosen among 0, 1, 2 and 3,
R₃ is such as defined above,
R₄ represents an atom of hydrogen; an atom of halogen; an alkyl, alkene or alkyne radical, comprising 1 to 18 carbons, possibly substituted by one or several amino, acid, acid derivative, alkoxy, aryl or hydroxy groups; an aryl radical, possibly substituted by one or several amino, acid, acid derivative, alkoxy, aryl or hydroxy groups; an arylalkyl or alkylaryl radical possibly substituted by one or several amino, acid, acid derivative, alkoxy, aryl or hydroxy groups; or an alkoxy radical possibly substituted by an aryl radical or a nitro function.

Still other compounds according to the invention comply with formula (III), or one of its pharmaceutically acceptable salts:

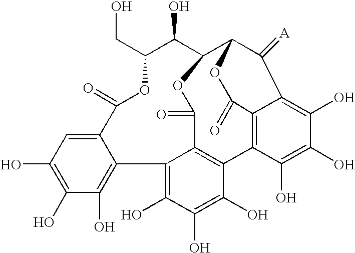

### Formula (III)

wherein:
A represents O, S, NOR₅, NR₆ or CR₅R₆,
R₅ represents an atom of hydrogen; an alkyl, alkene or alkyne radical, comprising 1 to 18 carbons, possibly substituted by one or several amino, acid, acid derivative, alkoxy, aryl or hydroxy groups; an aryl radical, possibly substituted by one or several amino, acid, acid derivative, alkoxy, aryl or hydroxy groups; an arylalkyl or alkylaryl radical possibly substituted by one or several amino, acid, acid derivative, alkoxy, aryl or hydroxy groups; or an alkoxy radical possibly substituted by an aryl radical, and
R₆ represents an alkyl, alkene or alkyne radical, comprising 1 to 18 carbons, possibly substituted by one or several amino, acid, acid derivative, alkoxy, aryl or hydroxy groups; an aryl radical, possibly substituted by one or several amino, acid, acid derivative, alkoxy, aryl or hydroxy groups; an arylalkyl or alkylaryl radical possibly substituted by one or several amino, acid, acid derivative, alkoxy, aryl or hydroxy groups; or an alkoxy radical possibly substituted by an aryl radical.

Halogen atom refers to an atom selected among fluorine, chlorine, bromine or iodine.

The alkyl, alkene or alkyne radicals may comprise 1 to 18 atoms of carbon, in particular 1 to 12 atoms of carbon, and in particular 1 to 6 atoms of carbon.

The alkene radicals may comprise one or several double bonds.

The alkyne radicals may comprise one or several triple bonds.

The aryl radicals may comprise 6 to 14 atoms of carbon and in particular 6 to 10 atoms of carbon.

The araylalkyl and alkylaryl radicals may comprise 7 to 25 atoms of carbon, in particular 7 to 20 atoms of carbon and in particular 7 to 15 atoms of carbon. In particular, the alkylaryl radical may represent a benzyl.

When R₁ and R₂ together form a cycle or a heterocycle, it may present 4 to 10 chains, in particular 6 to 8 chains.

In addition to atoms of carbon, the cycle in a heterocycle includes at least one heteroatom, selected among oxygen, nitrogen and sulphur.

One first group of preferred compounds according to the invention are those of formula (I) wherein m is 0 and R₁ represents an atom of hydrogen, n is 1, Y represents —O— and R₂ represents a group selected from the group comprising an atom of hydrogen and an alkyl radical comprising 1 to 18 atoms of carbon. In particular R₂ may represent a group chosen from the group comprising a atom of hydrogen and an alkyl radical comprising 1 to 6 atoms of carbon, and in particular a group selected from the group comprising a atom of hydrogen, the methyl, ethyl, propyl, butyl, pentyl and hexyl radical.

Another group of preferred compounds according to the invention are those in formula (I) wherein m is 1, X represents —O—, R₁ represents a group selected from the group comprising an atom of hydrogen and an alkyl radical comprising 1 to 18 atoms of carbon, n is 0, and R₂ represents an atom of hydrogen. In particular R₁ may represent a group chosen from the group comprising an atom of hydrogen and an alkyl radical comprising 1 to 6 atoms of carbon, and in particular a group selected from the group comprising an atom of hydrogen, the methyl, ethyl, propyl, butyl, pentyl and hexyl radical.

Still another group of preferred compounds according to the invention are those of formula (II) wherein p is 0 and R₄ represents a group selected from the group comprising an atom of hydrogen and an alkyl radical comprising 1 to 18 atoms of carbon. In particular R₄ may represent a group selected from the group comprising an atom of hydrogen and an alkyl radical comprising 1 to 6 atoms of carbon, and in particular a group selected from the group comprising an atom of hydrogen, the methyl, ethyl, propyl, butyl, pentyl and hexyl radical.

Another group of preferred compounds according to the invention are those of formula (III) in which A represents 0.

The invention also comprises specific compounds
Vescaline, corresponding to formula (I) in which m is 0, R₁ represents an atom of hydrogen, n is 1, Y represents —O—, and R₂ represents an atom of hydrogen,
Castaline, corresponding to formula (I) in which m is 1, X represents —O—, R₁ represents an atom of hydrogen, n is 0, and R₂ represents an atom of hydrogen, and
Vescalene, corresponding to formula (II) in which p is 0 and R₄ represents an atom of hydrogen.

The compounds of formula (I), (II) or (III) may if necessary be in solvate form, salt form or that of other physiologically acceptable derivatives. The salts and solvents that are acceptable for pharmaceutical use are generally those in which the counter-ion or the associated solvent is pharmaceutically acceptable.

The useable salts may be organic or mineral acids or bases. Among the acceptable acid salts, it can be mention those formed from hydrochloric, bromhydric, sulfuric, citric, tartric, phosphoric, lactic, pyruvic, acetic, trifluoroacetic, phenylacetic or triphenylacetic acid.

Among the acceptable alkaline salts, it can be mention the salts of alkaline metals, such as sodium or potassium, the salts of alcaline-earth metals, such as calcium and magnesium, and the salts formed from organic bases, such as the mono-, di- or tri-substituted amines.

The invention also refers to pharmaceutical compositions containing at least one compound mentioned above as an active agent.

In the pharmaceutical composition, the compounds are used in effective quantities. This is determined by the one skilled in the art, using different parameters, in particular with respect to the substance used, the age, weight, and physical condition of the patient, the mode of administration and the diet required. A doctor will be able to determine the mode of administration and the dosage for each patient.

The pharmaceutical composition may be administered in any form, topic or systemic, in particular in parenteral or enteral form.

When the composition or the drug are administered by enteral route, it may come in the form of tablets, capsules, sugar-coated tablets, syrups, suspensions, solutions, powders, granules, emulsions or microspheres.

In the case of parenteral administration, the composition may come in the form of solutions or suspensions for infusion or injection.

The composition comprises at least one pharmaceutically acceptable carrier.

It may also comprise at least one additive, in particular selected from among the colour, taste agents, and the preservatives. Of course, one skilled in the art will choose the additive(s), so that the beneficial properties intrinsically inherent in the invention are not, or are not substantially altered by the considered addition.

According to a specific variant, the composition comprises a concentration of ethanol less than 10% in weight, in particular less than 8% in weight, in particular less than 6% in weight, more specifically less than 4% in weight, and even more specifically less than 2% in weight with respect to the total weight of the composition, especially when said composition is void of ethanol.

According to another aspect, the invention also comprises the use of at least one compound of formula (I), (II) or (III) or one of their pharmaceutically acceptable salts, in the preparation of a composition intended to prevent or treat at least one disease involving an abnormal cell proliferation.

Said composition may be intended for human and/or veterinary medicine, and in particular it may be intended to treat or prevent at least one cancer chosen from among pancreatic cancer, oropharyngeal cancer, stomach cancer, cancer of the esophagus, colorectal cancer, brain cancer, in particular of the gliomas, ovarian cancer, cancer of the liver, cancer of the kidney, cancer of the larynx, cancer of the thyroid, lung cancer, bone cancer, the multiple myelomas, the mesotheliomas and the melanomas, skin cancer, breast cancer, prostatic cancer, bladder cancer, cancer of uterus, cancer of the testicles, non-Hodgkin lymphomas, leukemia, Hodgkin's disease, cancers of the soft tissue, as well as the secondary metastatic locations of the aforementioned cancers.

<<Abnormal proliferation >> refers to a proliferation that is independent of the normal mechanisms of control, for example the suspension of cell proliferation involving apoptosis (programmed cell death).

Another aspect of the invention includes the use of at least one compound of formula (I), (II) or (III) or one of their salts for the preparation of a pro-apoptotic composition.

Other advantages and characteristics of the invention appear in the following examples concerning the pro-apoptotic action and in which reference is made to the appended drawings where:

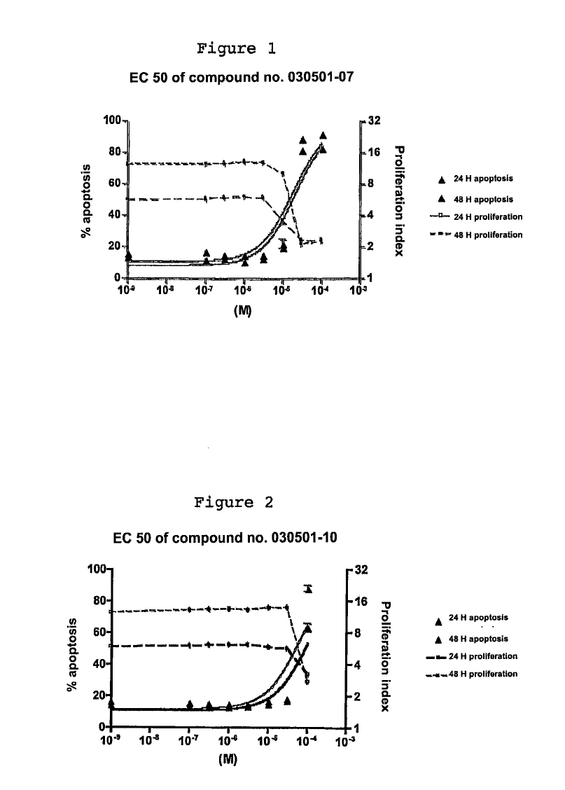

FIG. 1 represents the effect of different concentrations of Castaline on apoptosis at 24H and 48 H and cell proliferation at 24H and 48H.

FIG. 2 represents the effect of different concentrations of Vescalene on apoptosis at 24H and 48 H and cell proliferation at 24H and 48H.

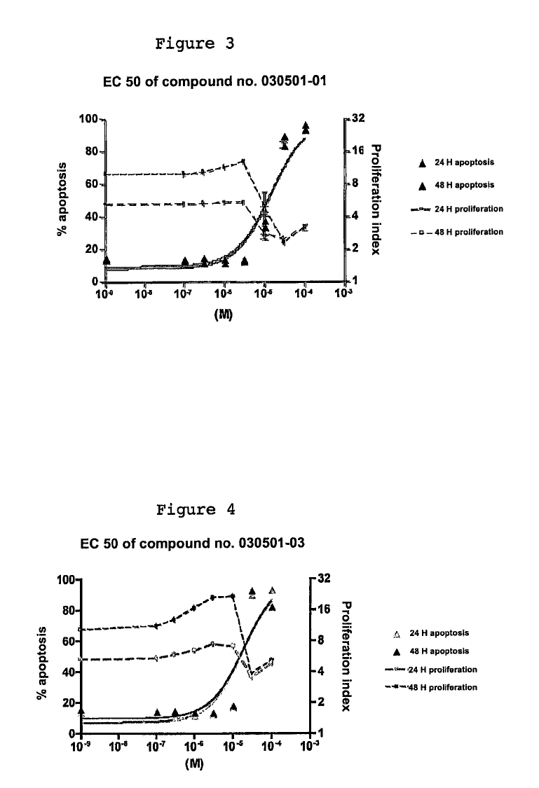

FIG. 3 represents the effect of different concentrations of Castalagine on apoptosis at 24H and 48H and cell proliferation at 24H and 48H.

FIG. 4 represents the effect of different concentrations of Acutissimine on apoptosis at 24H and 48H and cell proliferation at 24H and 48H.

The following examples are provided by way of indication and are in no way limiting.

### EXAMPLES

### Example 1

### Pro-Apoptotic Activity and Effect on Cell Proliferation of Castaline (Still Called Compound No. 030501-07)

FIG. 1 shows that Castaline has a pro-apototic activity and does not increase cell proliferation in a noteworthy manner with subtoxic doses.

### Example 2

### Pro-Apoptotic Activity and Effect on Cell Proliferation of Vescalene (Still Called Compound No. 030501-10)

FIG. 2 shows that Vescalene has a pro-apoptotic activity and does not increase cell proliferation in a noteworthy manner with subtoxic doses.

### Comparative Example 1

### Pro-Apoptotic Activity and Effect on Cell Proliferation of Castalagine (Still Called Compound No. 030501-01)

FIG. 3 shows that Castalagine increases cell proliferation with subtoxic doses, in particular in zone from 1 to 10 μm.

### Comparative Example 2

### Pro-Apoptotic Activity and Effect on Cell Proliferation of Acutissimine A (Still Called Compound No. 030501-03)

FIG. 4 shows that Acutissimine A increases cell proliferation with subtoxic doses, in particular in zone from 1 to 10 μM.

### Example 3

### Inhibition of DNA Topoisomerase II

| Compound (concentration) | Inhibition of decatenation (%) |
|---|---|
| Vescaline (1 μm) | 95.5 |
| Vescaline (10 μm) | 100 |
| Castaline (1 μm) | 87.9 |
| Castaline (10 μm) | 67.0 |
| Vescalene (1 μm) | 78.8 |
| Vescalene (10 μm) | 97.3 |

These results show the inhibiting action on topoisomerase II DNA of the compounds according to the invention.

1) A compound complying with formula (I), or one of its pharmaceutically acceptable salts, as a drug: 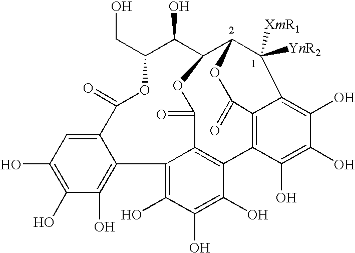
Formula (I)
wherein:
XₘR₁, and YₙR₂, identical or different, are present, or
one of XₘR₁, and YₙR₂ is absent, and in this case the bond between C₁ and C₂ is a double bond,
XₘR₁ and YₙR₂ together represent a group A and in this case the bond between C₁ and A is a double bond,
where:
R₁ and R₂ represent, independent of one another, an atom of hydrogen; an atom of halogen; an alkyl, alkene or alkyne radical, containing 1 to 18 atoms of carbon, possibly substituted by one or several amino, acid, acid derivative, alkoxy, aryl or hydroxy groups; an aryl radical possibly substituted by one or several amino, acid, acid derivative, alkoxy, aryl or hydroxy groups; an arylalkyl or alkylaryl radical, possibly substituted by one or several amino, acid, acid derivative, alkoxy, aryl or hydroxy groups; or a nitro function;
or R₁ and R₂ together form a cycle or a heterocycle, possibly substituted,
m is a whole number chosen among 0 and 1, n is a whole number chosen among 0 and 1,
X and Y represent independent of each other —O—, —OC(═O)—, —OC(═O)O—, —OC(═O)N—, —NR₃—, —NR₃C(═O)—, —NR₃C(═O)O—, —NR₃C(O)NR₃—, —C(═O)—, —C(═O)O—, —S(O)_{q}—, or —P(O)ᵣ—,
q is a whole number chosen among 0, 1, 2 and 3, r is a whole number chosen from among 0, 1, 2 and 3,
R₃ represents an atom of hydrogen; an alkyl, alkene or alkyne radical, comprising 1 to 18 carbons, possibly substituted by one or several amino, acid, acid derivative, alkoxy, aryl or hydroxy groups; an aryl radical, possibly substituted by one or several amino, acid, acid derivative, alkoxy, aryl or hydroxy groups; an arylalkyl or alkylaryl radical possibly substituted by one or several amino, acid, acid derivative, alkoxy, aryl or hydroxy groups; or an alkoxy radical possibly substituted by an aryl radical;
A represents O, S, NOR₅, NR₆ or CR₅R₆, where R₅ and R₆ represent, independent of each other, an atom of hydrogen, an alkyl, alkene or alkyne radical, comprising 1 to 18 atoms of carbon, possibly substituted by one or several amino, acid, acid derivative, alkoxy, aryl or hydroxy groups; an arylalkyl or alkylaryl radical possibly substituted by one or several amino, acid, acid derivative, alkoxy, aryl or hydroxy groups; or an alkoxy radical, possibly substituted by an aryl radical.

2) A compound according to claim 1, characterised in that it complies with the following formula (I): 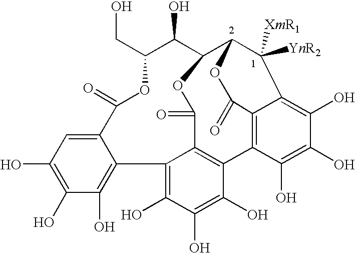
Formula (I)
wherein:
R₁ and R₂, independent of one another, represents an atom of hydrogen; an atom of halogen; an alkyl, alkene or alkyne radical, containing 1 to 18 atoms of carbon, possibly substituted, by one or several amino, acid, acid derivative, alkoxy, aryl or hydroxy groups; an aryl radical possibly substituted by one or several amino, acid, acid derivative, alkoxy, aryl or hydroxy groups; an arylalkyl or alkylaryl radical, possibly substituted by one or several amino, acid, acid derivative, alkoxy, aryl or hydroxy groups; or a nitro function;
or R₁ and R₂ together form a cycle or a heterocycle, possibly substituted,
m is a whole number chosen from among 0 and 1, n is a whole number chosen from among 0 and 1,
X and Y represent independent of each other —O—, —OC(—O)—, —OC(═O)O—, —OC(═O)N—, —NR₃—, —NR₃C(═O)—, —NR₃C(═O)O—, —NR₃C(O)NR₃—, —C(═O)—, —C(═O)O—, —S(O)_{q}—, or —P(O)ᵣ—,
q is a whole number chosen from among 0, 1, 2 and 3, r is a whole number chosen from among 0, 1, 2 and 3,
R₃ represents an atom of hydrogen; an alkyl, alkene or alkyne radical, comprising from 1 to 18 carbons, possibly substituted by one or several amino, acid, acid derivative, alkoxy, aryl or hydroxy groups; an aryl radical, possibly substituted by one or several amino, acid, acid derivative, alkoxy, aryl or hydroxy groups; an arylalkyl or alkylaryl radical possibly substituted by one or several amino, acid, acid derivative, alkoxy, aryl or hydroxy groups; or an alkoxy radical possibly substituted by an aryl radical.

3) A compound according to claim 2, characterised in that it complies with formula (I) wherein m is 0 and R₁ represents an atom of hydrogen, n is 1, Y represents —O— and R₂ represents a group selected from the group comprising an atom of hydrogen and an alkyl radical comprising 1 to 18 atoms of carbon.

4) A compound according to claim 2, characterised in that it complies with formula (I) wherein m is 1, x represents —O—, R₁ represents a group selected from the group comprising an atom of hydrogen and an alkyl radical comprising 1 to 18 atoms of carbon, n is 0, and R₂ represents an atom of hydrogen.

5) A compound according to claim 1, characterised in that it complies with formula (II): 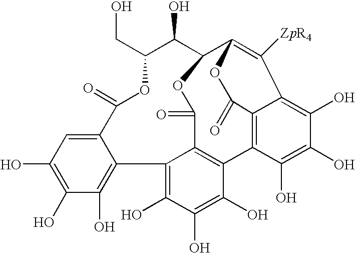
Formula (II)
where:
p is a whole number selected from among 0 and 1,
Z represents —O—, —OC(═O)—, —OC(═O)O—, —OC(═O)N—, —NR₃—, —NR₃C(═O)—, —NR₃C(═O)O—, —NR₃C(O)NR₃—, —C(═O)—, —C(═O)O—, —S(O)_{q}—, or —P(O)ᵣ—,
q is a whole number selected from among 0, 1, 2 and 3, r is a whole number selected from among 0, 1, 2 and 3,
R₃ represents an atom of hydrogen; an alkyl, alkene or alkyne radical, comprising 1 to 18 carbons, possibly substituted by one or several amino, acid, acid derivative, alkoxy, aryl or hydroxy groups; an arylalkyl or alkylaryl radical possibly substituted by one or several amino, acid, acid derivative, alkoxy, aryl or hydroxy groups; or an alkoxy radical possibly substituted by an aryl radical, and
R₄ represents an atom of hydrogen; an atom of halogen; an alkyl, alkene or alkyne radical,
comprising 1 to 18 carbons, possibly substituted by one or several amino, acid, acid derivative, alkoxy, aryl or hydroxy groups; an aryl radical, possibly substituted by one or several amino, acid, acid derivative, alkoxy, aryl or hydroxy groups; an arylalkyl or alkylaryl radical possibly substituted by one or several amino, acid, acid derivative, alkoxy, aryl or hydroxy groups; or an alkoxy radical possibly substituted by an aryl radical or a nitro function.

6) A compound according to claim 5, characterised in that it complies with formula (II) in which p is 0 and R₄ represents a group selected from the group comprising an atom of hydrogen and an alkyl radical comprising 1 to 18 atoms of carbon.

7) A compound according to claim 1, characterised in that it complies with formula (III): 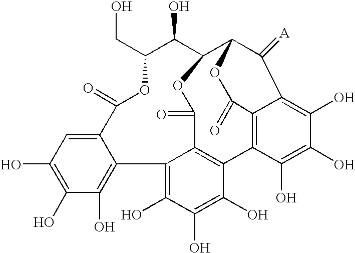
Formula (III)
wherein:
A represents O, S, NOR₅, NR₆ or CR₅R₆,
R₅ represents an atom of hydrogen; an alkyl, alkene or alkyne radical, comprising 1 to 18 atoms of carbon, possibly substituted by one or several amino, acid, acid derivative, alkoxy, aryl or hydroxy groups; an aryl radical, possibly substituted by one or several amino, acid, acid derivative, alkoxy, aryl or hydroxy groups; an arylalkyl or alkylaryl radical possibly substituted by one or several amino, acid, acid derivative, alkoxy, aryl or hydroxy groups; or an alkoxy radical possibly substituted by an aryl radical, and
R₆ represents an alkyl, alkene or alkyne radical, comprising 1 to 18 atoms of carbon, possibly substituted by one or several amino, acid, acid derivative, alkoxy, aryl or hydroxy groups; an aryl radical, possibly substituted by one or several amino, acid, acid derivative, alkoxy, aryl or hydroxy groups; an arylalkyl or alkylaryl radical possibly substituted by one or several amino, acid, acid derivative, alkoxy, aryl or hydroxy groups; or an alkoxy radical possibly substituted by an aryl radical.

8) A compound according to claim 1, characterised in that it complies with formula (III) wherein A represents 0.

9) The use of at least one compound of formula (I), (II) or (III) as defined according to any of claims 1 to 8 in the preparation of a composition intended to prevent or treat at least one disease involving abnormal cell proliferation in particular cancer, and in particular a cancer selected from among pancreatic cancer, oropharyngeal cancer, stomach cancer, cancer of the esophagus, colorectal cancer, brain cancer, in particular of the gliomas, ovarian cancer, cancer of the liver, cancer of the kidney, cancer of the larynx, cancer of the thyroid, lung cancer, bone cancer, the multiple myelomas, the mesotheliomas and the melanomas, skin cancer, breast cancer, prostatic cancer, bladder cancer, cancer of uterus, cancer of the testicles, non-Hodgkin lymphomas, leukemia, Hodgkin's disease, cancers of the soft tissue, as well as the secondary metastatic locations of the aforementioned cancers.

10) A pharmaceutical composition comprising by way of an active agent at least one compound as defined according to any of claims 1 to 8 and at least one pharmaceutically acceptable carrier.

11) The use of at least one compound as defined according to any of claims 1 to 8 for the preparation of a pro-apoptotic composition.
